(19) United States
(12) Patent Application Publication
(10) Pub. No.: US 2006/0045886 A1
(43) Pub. Date: Mar. 2, 2006

(54) HIV immunostimulatory compositions

(76) Inventor: Ross M. Kedl, Denver, CO (US)

Correspondence Address: 3M INNOVATIVE PROPERTIES COMPANY, PO BOX 33427, ST. PAUL, MN, 55133-3427, US

(21) Appl. No.: 11/213,405

(22) Filed: Aug. 26, 2005

Related U.S. Application Data
(60) Provisional application No. 60/604,903, filed on Aug. 27, 2004.

(51) Int. Cl. A61K 39/21; A61K 31/4745

(52) U.S. Cl. 424/188.1; 514/292

(57) ABSTRACT

The present invention provides an IRM-HIV composition that includes an IRM portion paired with an HIV antigenic portion.

### CROSS-REFERENCE TO RELATED APPLICATION

This application claims priority to U.S. Provisional Patent Application Ser. No. 60/604,903, filed Aug. 27, 2004.

### BACKGROUND

Immune response modifiers (“IRMs”) include compounds that possess potent immunomodulating activity including but not limited to antiviral and antitumor activity. Certain IRMs modulate the production and secretion of cytokines. For example, certain IRM compounds induce the production and secretion of cytokines such as, e.g., Type I interferons, TNF-α, IL-1, IL-6, IL-8, IL-10, IL-12, MIP-1, and/or MCP-1. As another example, certain IRM compounds can inhibit production and secretion of certain T_{H}2 cytokines, such as IL-4 and IL-5. Additionally, some IRM compounds are said to suppress IL-1 and TNF (U.S. Pat. No. 6,518,265).

Certain IRMs are small organic molecules (e.g., molecular weight under about 1000 Daltons, preferably under about 500 Daltons, as opposed to large biological molecules such as proteins, peptides, and the like) such as those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; 6,797,718; and 6,818,650; U.S. patent Publication Nos. 2004/0091491; 2004/0147543; and 2004/0176367; and International Publication Nos. WO 2005/18551, WO 2005/18556, and WO 2005/20999.

Additional examples of small molecule IRMs include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08905), and certain 3-β-D-ribofuranosylthiazolo[4,5-d]pyrimidine derivatives (such as those described in U.S. Publication No. 2003/0199461).

Other IRMs include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG sequences and are described, for example, in International Patent Publication No. WO 00/75304.

Other IRMs include biological molecules such as aminoalkyl glucosaminide phosphates (AGPs) and are described, for example, in U.S. Pat. Nos. 6,113,918; 6,303,347; 6,525,028; and 6,649,172.

Certain IRMs can function as Toll-like receptor (TLR) agonists. Some small molecule IRMs may act through one or more of TLRs 2, 4, 6, 7, and 8. CpG may act through TLR 9.

By stimulating certain aspects of the immune system, as well as suppressing other aspects (see, e.g., U.S. Pat. Nos. 6,039,969 and 6,200,592), IRMs may be used to treat many diseases. For example, the small molecule IRM imiquimod is useful for the treatment of external genital and perianal warts caused by human papillomavirus, actinic keratosis, and basal cell carcinoma. Examples of other diseases that may be treated using IRMs include, but are not limited to, eczema, essential thrombocythaemia, hepatitis B, multiple sclerosis, other neoplastic diseases, psoriasis, rheumatoid arthritis, type I herpes simplex, and type II herpes simplex.

IRM compounds also can modulate humoral immunity by stimulating antibody production by B cells. Further, various IRMs have been shown to be useful as vaccine adjuvants (see, e.g., U.S. Pat. Nos. 6,083,505 and 6,406,705).

### SUMMARY OF THE INVENTION

It has now been found that IRMs, especially small molecule IRMs and agonists of TLR7 and/or TLR8, are surprisingly effective at stimulating an immune response when chemically or physically paired with certain Human Immunodeficiency Virus (HIV) antigens to form an immunostimulatory composition. The immunostimulatory effect of a particular composition may be greater than the immunostimulatory effect of the same HIV antigen and the same or a comparable IRM as that in the composition, but administered in an unpaired form.

The present invention provides an immunostimulatory composition that includes an IRM portion paired with an HIV antigenic portion. In some embodiments, the IRM portion may be, or be derived from, an agonist of TLR7 and/or TLR8. In other embodiments, the IRM portion may include, or be derived from, an imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, pyrazoloquinoline amine, a tetrahydropyrazoloquinoline amine, a pyrazolonaphthyridine amine, or a tetrahydropyrazolonaphthyridine amine. The antigenic portion may be, or be derived from, a Gag protein or polyprotein, an Env protein or polyprotein, a Pol protein or polyprotein, Nef, Pro, Rev, Tat, Vif, Vpr, Vpx, or an antigenic fragment thereof Furthermore, the form of the antigenic portion may be a protein, a peptide, a lipoprotein, or a glycoprotein.

### BRIEF DESCRIPTION OF THE DRAWINGS

FIG. 1a-1c shows the generation of a T_{H}1 and CTL response after immunization with an IRM-HIV composition.

FIG. 2 shows the generation of IFN-γ producing cells after immunization with an IRM-HIV composition.

FIG. 3 shows the generation of IL-2 producing cells after immunization with an IRM-HIV composition.

FIG. 4a-b shows the generation of a T_{H}1 and CTL response after immunization with an IRM-HIV composition.

FIG. 5 shows HIV Gag-specific antibody titers in serum after immunization with an IRM-HIV composition.

### DETAILED DESCRIPTION OF ILLUSTRATIVE EMBODIMENTS OF THE INVENTION

The present invention provides immunostimulatory compositions that include an IRM portion paired with an HIV antigenic portion. These IRM-HIV compositions can provide an even greater immune response than compositions containing the same or a comparable IRM and the same HIV antigen, but in an unpaired form. In some aspects, eliciting an immune response with an IRM-HIV composition may provide effective treatment against infection with HIV. The IRM-HIV compositions may be designed to elicit a cell-mediated immune response, a humoral immune response, or both.

Also provided are methods of making IRM-HIV compositions, methods of eliciting an immune response using IRM-HIV compositions, and methods of enhancing anti-HIV immunostimulatory activity of an IRM with an HIV antigen.

For purposes of this invention, the following terms shall have the meanings set forth as follows:

“Agonist” refers to a compound that can combine with a receptor (e.g., a TLR) to induce a cellular activity. An agonist may be a ligand that directly binds to the receptor. Alternatively, an agonist may combine with a receptor indirectly by, for example, (a) forming a complex with another molecule that directly binds to the receptor, or (b) otherwise results in the modification of another compound so that the other compound directly binds to the receptor. An agonist may be referred to as an agonist of a particular TLR (e.g., a TLR7 agonist) or a particular combination of TLRs (e.g., a TLR 7/8 agonist—an agonist of both TLR7 and TLR8).

“Antigen” refers to any substance that is capable of being the target of an immune response. An antigen may be the target of, for example, a cell-mediated and/or humoral immune response raised by a subject organism. Alternatively, an antigen may be the target of a cellular immune response (e.g., immune cell maturation, production of cytokines, production of antibodies, etc.) when contacted with immune cells.

“Paired” and variations thereof refer to components associated in some chemical or physical manner so that the components are not freely dispersible from one another, at least until contacting an immune cell. For example, two components may be covalently bound to one another so that the two components are incapable of separately dispersing or diffusing. Pairing also may be achieved by, for example, non-covalent affinity binding, ionic binding, hydrophilic or hydrophobic affinity, physical entrapment (e.g., within a liposome), and the like. Pairing is specifically distinguished from a simple mixture of antigen and adjuvant such as may be found, for example, in a conventional vaccine. In a simple mixture, the components can be free to independently disperse within the vaccinated environment. As used herein, “paired” and variations thereof refer to components that maintain a chemical or physical association after immunization at least until they contact an immune cell.

“Polypeptide” refers to a sequence of amino acid residues without regard to the length of the sequence. Therefore, the term “polypeptide” refers to any amino acid sequence having at least two amino acids and includes full-length proteins and, as the case may be, polyproteins.

“Treat” or variations thereof refer to reducing, limiting progression, ameliorating, or resolving, to any extent, the symptoms or signs related to a condition. A treatment may be “therapeutic” which, as used herein, refers to a treatment that ameliorates one or more existing symptoms or clinical signs associated with a condition. Alternatively, a treatment may be “prophylactic” which, as used herein, refers to a treatment that limits, to any extent, the development and/or appearance of a symptom or clinical sign of a condition.

Also, any recitation of a numerical range by endpoints includes all numbers subsumed within that range (e.g., 1 to 5 includes 1, 1.5, 2, 2.75, 3, 3.80, 4, 5, etc.).

Unless otherwise indicated, reference to a compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

The IRM portion of an IRM-HIV composition may be, or be derived from, any suitable IRM compound. Suitable IRM compounds include small organic molecules, i.e., molecules having a molecular weight of less than about 1000 Daltons, although in some embodiments the IRM may have a molecular weight of less than about 700 Daltons and in some cases the IRM may have a molecular weight from about 500 Daltons to about 700 Daltons.

In some embodiments, a suitable IRM compound can include, but is not limited to, a small molecule IRM compound such as those described above or a derivative thereof. Suitable small molecule IRMs, having a 2-aminopyridine fused to a five membered nitrogen-containing heterocyclic ring, include but are not limited to imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, hydroxylamine substituted imidazoquinoline amines, oxime substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, hydroxylamine substituted tetrahydroimidazoquinoline amines, oxime substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; pyrazolopyridine amines; pyrazoloquinoline amines; tetrahydropyrazoloquinoline amines; pyrazolonaphthyridine amines; tetrahydropyrazolonaphthyridine amines; and 1H-imidazo dimers fused to pyridine amines, quinoline amines, tetrahydroquinoline amines, naphthyridine amines, or tetrahydronaphthyridine amines.

In some embodiments, the IRM portion can include, or be derived from, an imidazoquinoline amine such as, for example, a substituted imidazoquinoline amine or an amide substituted imidazoquinoline amine. In certain specific embodiments, the IRM portion may include, or be derived from, a substituted imidazoquinoline amine such as, for example, 1-(2-amino-2-methylpropyl)-2-(ethoxymethyl)-1H-imidazo[4,5-c]quinolin-4-amine. In other embodiments, the IRM portion may include, or be derived from, an amide substituted imidazoquinoline amine such as, for example, N-[6-({2-[4-amino-2-(ethoxymethyl)-1H-imidazo[4,5-c]quinolin-1-yl]-1,1-dimethylethyl}amino)-6-oxohexyl]-4-azido-2-hydroxybenzamide.

Additional suitable small molecule IRMs include the certain purine derivatives, certain imidazoquinoline amide derivatives, certain benzimidazole derivatives, and certain derivatives of a 4-aminopyrimidine fused to a five membered nitrogen containing heterocyclic ring (e.g., adenine derivatives) described above.

Other suitable IRMs include the CpGs and other IRM nucleotide sequences that lack CpG described above.

In some embodiments, the IRM portion may include, or be derived from, an agonist of one or more of TLRs 2, 4, 6, 7, 8 and 9. In certain embodiments, the IRM portion includes an agonist of TLR7. In other embodiments, the IRM portion includes an agonist of TLR8. In certain particular embodiments the IRM portion includes an agonist of both TLR7 and TLR8 (i.e., a TLR7/8 agonist).

In some embodiments, the IRM portion of an IRM-HIV composition may include a combination of two or more IRMs, if desired.

The HIV antigenic portion can include, or be derived from, any material that raises a cell-mediated immune response, a humoral immune response, or both, against at least a portion of the Human Immunodeficiency Virus (HIV). Suitable antigenic material can include, for example, an HIV protein, an HIV polyprotein, or an antigenic polypeptide fragment of any HIV protein or HIV polyprotein.

Two types of HIV have been identified, HIV-1 and HIV-2. Both HIV-1 and HIV-2 have the same modes of transmission and are associated with similar opportunistic infections and conditions. However, immunodeficiency develops more slowly and is milder in persons infected with HIV-2 than that in persons infected with HIV-1. The geographic distributions of HIV-1 and HIV-2 differ markedly. HIV-1 is found in relative abundance throughout the world and is responsible for the global HIV pandemic, whereas the geographic distribution of HIV-2 is much more limited. HIV-2 is found primarily in west Africa and several other African countries, with additional documented infections in Europe, Asia, and, although rare, North America. In all regions, the proportion of HIV-1 infections is considerably larger than that of HIV-2 infections.

The HIV antigenic portion of an IRM-HIV composition can include, or be derived from, any antigenic portion of HIV-1. Suitable HIV-1 antigens can include, for example, a Group-specific antigen (i.e., Gag) protein or polyprotein such as, for example p17 (a matrix protein), p24 (a capsid protein), p7 (a nucleocapsid protein), p6 (a Vpr binding protein), p55 (a precursor polyprotein), and p2 and p1; an Envelope (Env) protein or polyprotein such as, for example gp120 (a surface protein), gp41 (a transmembrane protein), and gp160 (a precursor protein); a Pol protein or polyprotein such as, for example, p15 (a protease), p51 (reverse transcriptase), p15 (RNase H), p66 (RNase H+reverse transcriptase), and p31 (integrase); Gag-Pol polyprotein (p160); Viral protein R (Vpr, p12/p10); Virion Infectivity Factor (Vif, p23); Transactivating regulatory protein (Tat, p16/p14); ART/TRS Anti-repression transactivator protein (Rev, p19); Negative Factor (Nef, p27/p25); or Viral protein U, (Vpu, p16).

Alternatively, the HIV antigenic portion of an IRM-HIV composition can include, or be derived from, any antigenic portion of HIV-2. Suitable HIV-2 antigens include, for example, a Group-specific antigen (i.e., Gag) protein or polyprotein such as, for example p17 (a matrix protein), p24 (a capsid protein), p7 (a nucleocapsid protein), p6 (a Vpr binding protein), p55 (a precursor polyprotein), and p2 and p1; an Envelope (Env) protein or polyprotein such as, for example gp120 (a surface protein), gp41 (a transmembrane protein), and gp160 (a precursor protein); a Pol protein or polyprotein such as, for example, p15 (a protease), p51 (reverse transcriptase), p15 (RNase H), p66 (RNase H+reverse transcriptase), and p31 (integrase); Gag-Pol polyprotein (p160); Viral protein R (Vpr, p12/p10); Virion Infectivity Factor (Vif, p23); Transactivating regulatory protein (Tat, p16/p14); ART/TRS Anti-repression transactivator protein (Rev, p19); Negative Factor (Nef, p27/p25); or Viral protein X (Vpx, p16/p12).

In some embodiments, the HIV antigenic portion may include, or be derived from, a Gag protein. In certain specific embodiments, the HIV antigenic portion may be, or be derived from, Gag p24. In other embodiments, the HIV antigenic portion may be, or be derived from, Gag p41.

In some embodiments, the HIV antigenic portion of an IRM-HIV composition may include a combination of two or more HIV antigens, if desired. In embodiments that include a combination of two or more HIV antigens, the HIV antigenic portion can include two or more related HIV antigens (e.g., two or more Gag proteins, two or more Env proteins, two or more Pol proteins, etc.) or two or more unrelated HIV antigens (e.g., at least one Gag protein and at least one Pol protein, at least on Env protein and Nef, etc.).

In some embodiments, the IRM-HIV composition includes an amide substituted imidazoquinoline amine such as, for example, N-[6-({2-[4-amino-2-(ethoxymethyl)-1H-imidazo[4,5-c]quinolin-1-yl]-1,1-dimethylethyl}amino)-6-oxohexyl]-4-azido-2-hydroxybenzamide as the IRM portion and Gag p24 as the HIV antigenic portion. In other embodiments, the IRM-HIV composition includes an amide substituted imidazoquinoline amine such as, for example, N-[6-({2-[4-amino-2-(ethoxymethyl)-1H-imidazo[4,5-c]quinolin-1-yl]-1,1-dimethylethyl}amino)-6-oxohexyl]-4-azido-2-hydroxybenzamide as the IRM portion and Gag p41 as the HIV antigenic portion. In one specific embodiment, the IRM-HIV composition includes N-[6-({2-[4-amino-2-(ethoxymethyl)-1H-imidazo[4,5-c]quinolin-1-yl]-1,1-dimethylethyl}amino)-6-oxohexyl]-4-azido-2-hydroxybenzamide covalently conjugated to Gag p24. In an alternative embodiment, the IRM-HIV composition includes N-[6-({2-[4-amino-2-(ethoxymethyl)-1H-imidazo[4,5-c]quinolin-1-yl]-1,1-dimethylethyl}amino)-6-oxohexyl]-4-azido-2-hydroxybenzamide covalently conjugated to Gag p41.

An IRM-HIV composition includes an effective amount of biological activity of both the IRM portion and the HIV antigenic portion. An effective amount of biological activity of the IRM portion (“IRM activity”) includes one or more of the following: an increase in cytokine production by T cells, activation of T cells specific to the HIV antigenic portion, and activation of dendritic cells. An effective amount of biological activity of the HIV antigenic portion (“HIV activity”) includes one or more of the following: generation of antibodies specific to the HIV antigenic portion by B cells and generation of antigen-presenting cells (APCs) that present the HIV antigenic portion. An IRM-HIV composition may be combined with a pharmaceutically acceptable carrier, one or more excipients, or some combination of the foregoing in order to form a pharmaceutical composition.

An IRM-HIV composition may be provided in any formulation suitable for administration to a subject. Suitable types of formulations are described, for example, in U.S. Pat. No. 5,736,553; U.S. Pat. No. 5,238,944; U.S. Pat. No. 5,939,090; U.S. Pat. No. 6,365,166; U.S. Pat. No. 6,245,776; U.S. Pat. No. 6,486,168; European Patent No. EP 0 394 026; and U.S. patent Publication No. 2003/0199538. A suitable formulation may be, for example, a solution, a suspension, an emulsion, or any form of mixture. An IRM-HIV composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including but not limited to adjuvants, skin penetration enhancers, colorants, fragrances, flavorings, moisturizers, thickeners, and the like.

A formulation containing an E?M-HIV composition may be administered in any suitable manner such as, for example, non-parenterally or parenterally. As used herein, non-parenterally refers to administration through the digestive tract, including by oral ingestion. Parenterally refers to administration other than through the digestive tract such as, for example, intravenously, intramuscularly, transdermally, subcutaneously, transmucosally (e.g., by inhalation), or topically.

The composition of a formulation suitable for administering to a subject may vary according to factors known in the art including but not limited to the physical and chemical nature of the IRM-HIV composition, the nature of the carrier, the intended dosing regimen, the state of the subject's immune system (e.g., suppressed, compromised, stimulated), the method of administering the IRM-HIV composition, and the species to which the formulation is being administered. Accordingly, it is not practical to set forth generally the composition of a formulation effective for use as an HIV vaccine. Those of ordinary skill in the art, however, can readily determine an appropriate formulation with due consideration of such factors.

In some embodiments, the IRM-HIV composition may be administered to a subject in a formulation of, for example, from about 0.0001% to about 10% (unless otherwise indicated, all percentages provided herein are weight/weight with respect to the total formulation) to the subject, although in some embodiments the IRM-HIV composition may be administered using a formulation that provides IRM-HIV composition in a concentration outside of this range. In some embodiments, the IRM-HIV composition may be administered in a formulation that includes at least about 0.01%, at least about 0.05%, at least about 0.1%, at least about 0.5%, at least about 1%, or even at least about 5% IRM-HIV composition. In some embodiments, the IRM-HIV composition may be administered in a formulation that includes no more than about 10%, no more than about 5%, no more than about 1%, no more than about 0.5%, or even no more than about 0.1% IRM-HIV composition. In one particular embodiment, the IRM-HIV composition may be administered in a formulation that includes from about 0.1% IRM-HIV composition to about 5% IRM-HIV composition.

An amount of an IRM-HIV composition effective for eliciting an immune response against an HIV antigen is an amount sufficient to induce at least a biological response associated with a THl immune response or a CTL immune response. The precise amount of IRM-HIV composition necessary to be an effective amount may vary according to factors known in the art including but not limited to the physical and chemical nature of the IRM-HIV composition, the nature of the carrier, the intended dosing regimen, the state of the subject's immune system (e.g., suppressed, compromised, stimulated), the method of administering the IRM-HIV composition, and the species to which the IRM-HIV composition is being administered. Accordingly, it is not practical to set forth generally the amount that constitutes an amount of IRM-HIV composition effective to elicit an immune response against an HIV antigen for all possible situations. Those of ordinary skill in the art, however, can readily determine the appropriate amount with due consideration of such factors.

In some embodiments, the methods of the present invention include administering sufficient IRM-HIV composition to provide a dose of, for example, from about 100 ng/kg to about 50 mg/kg to the subject, although in some embodiments the methods may be performed by administering IRM-HIV composition in a dose outside this range. In some embodiments, the IRM-HIV composition may be administered to provide a dose of at least about 100 ng/kg, at least about 1 μg/kg, at least about 30 μg/kg, at least about 100 μg/kg, at least about 300 μg/kg, or even 1 mg/kg. In some embodiments, the IRM-HIV composition may be administered to provide a dose of no more than 50 mg/kg, no more than 10 mg/kg, no more than 5 mg/kg, no more than 1 mg/kg, no more than 500 μg/kg, no more than 100 μg/kg, or even no more than 50 μg/kg. In one particular embodiment, the IRM-HIV composition may be administered to provide a dose of from about 30 μg/kg IRM-HIV composition to about 500 μg/kg IRM-HIV composition, such as, for example, a dose of about 30 μg/kg, 40 μg/kg, 50 μg/kg, 66 μg/kg, or 400 μg/kg.

An IRM-HIV composition may be administered to a patient in order to provide treatment to the patient against infection by HIV. The treatment may be intended to be prophylactic—e.g., the IRM-HIV composition may be administered to a patient that has not developed any symptoms or clinical signs of HIV infection. In such cases, administering the IRM-HIV composition to the patient may decrease the likelihood and/or extent to which the patient develops symptoms or clinical signs of HIV infection in the event that the patient is subsequently exposed to HIV. Alternatively, the treatment may be intended to be therapeutic—e.g., the IRM-HIV composition may be administered to one who has already developed symptoms or clinical signs of HIV infection. In such cases, administering the IRM-HIV composition to the patient may slow the progression of the infection, limit, reduce or even resolve the infection, thereby slowing, reducing, limiting the severity of, or preventing symptoms or clinical signs of HIV infection, including symptoms or clinical signs of secondary conditions associated with HIV infection.

An IRM-HIV composition can be administered as the single therapeutic agent in a treatment regimen. Alternatively, an IRM-HIV composition may be administered in combination with another pharmaceutical composition or with other active agents, including additional IRMs, antivirals, antibiotics, antibodies, proteins, peptides, oligonucleotides, etc.

An IRM-HIV composition can be administered once or in a treatment regimen that includes a plurality of administrations. The precise number, frequency, and duration of a treatment regimen may vary according to factors known in the art including but not limited to the physical, pharmacological, and chemical nature of the IRM-HIV composition, the state of the subject's immune system (e.g., suppressed, compromised, stimulated), the method of administering the IRM-HIV composition, and the desired effect (e.g., prophylactic vs. therapeutic), and the species to which the IRM-HIV composition is being administered. Accordingly, it is not practical to set forth generally the amount that constitutes an amount of IRM-HIV composition effective to elicit an immune response against an HIV antigen for all possible situations. Those of ordinary skill in the art, however, can readily determine the appropriate amount with due consideration of such factors.

In some embodiments, the IRM-HIV composition may be administered only once. In other embodiments, the treatment regimen may include one or more booster immunizations. Booster immunizations may be provided at regular intervals or on an “as needed” basis. A regular interval may be days, weeks, months, or years in duration. Accordingly, booster immunizations may be administered, for example, every two weeks, every three weeks, every four weeks, every three months, every six months, every year, every five years, or every ten years.

In some embodiments, the IRM portion of the composition may be covalently coupled to the HIV antigenic portion to form an IRM-HIV conjugate. As used herein, “covalently coupled” refers to direct and/or indirect coupling of two components exclusively through covalent bonds. Direct covalent coupling may involve direct covalent binding between an atom of the IRM portion and an atom of the HIV antigenic portion. Alternatively, the covalent coupling may occur through a linking group covalently attached to the IRM portion, the HIV antigenic portion, or both, that facilitates covalent coupling of the IRM portion and the HIV antigenic portion. Indirect covalent coupling may include a third component such as, for example, a solid support to which both the IRM portion and the HIV antigenic portion are separately covalently attached. Also, “covalently coupled” and “covalently attached” are used interchangeably.

An IRM-HIV conjugate can include an IRM moiety as the IRM portion and an HIV antigen-containing moiety as the HIV antigenic portion. When synthesizing an IRM-HIV conjugate, each of the IRM moiety, the linking group, and the HIV antigen-containing moiety may be selected so that the resulting IRM-HIV conjugate possesses an effective amount of IRM activity and an effective amount of HIV antigenic activity.

The linking group can be any suitable organic linking group that allows the HIV antigen-containing moiety to be covalently coupled to the IRM moiety while preserving an effective amount of IRM activity and HIV antigenic activity. In some embodiments, the linking group may be selected to create sufficient space between the active core of the IRM moiety and the HIV antigen-containing moiety that the HIV antigen-containing moiety does not interfere with a biologically effective interaction between the IRM moiety and antigen presenting cells that results in IRM activity such as, for example, cytokine production.

The linking group includes a reactive group capable of reacting with the antigen to form a covalent bond. Suitable reactive groups include those discussed in Hermanson, G. (1996), Bioconjugate Techniques, Academic Press, Chapter 2 “The Chemistry of Reactive Functional Groups”, 137-166. For example, the linking group may react with a primary amine (e.g., an N-hydroxysuccinimidyl ester or an N-hydroxysulfosuccinimidyl ester); it may react with a sulfhydryl group (e.g., a maleimide or an iodoacetyl), or it may be a photoreactive group (e.g. a phenyl azide including 4-azidophenyl, 2-hydroxy-4-azidophenyl, 2-nitro-4-azidophenyl, and 2-nitro-3-azidophenyl).

A chemically active group accessible for covalent coupling to the linking group includes groups that may be used directly for covalent coupling to the linking group or groups that may be modified to be available for covalent coupling to the linking group. For example, suitable chemically active groups include but are not limited to primary amines and sulfhydryl groups. Because certain HIV antigen-containing moieties, e.g., proteins and other peptides, may include a plurality of chemically active groups, certain IRM-HIV conjugates may include a plurality of IRM moieties conjugated to a particular HIV antigen-containing moiety.

IRM-HIV conjugates generally may be prepared by reacting an IRM with a crosslinker and then reacting the resulting intermediate with an HIV antigen. Many crosslinkers suitable for preparing bioconjugates are known and many are commercially available. See for example, Hermanson, G. (1996) Bioconjugate Techniques, Academic Press.

IRM-HIV conjugates may be prepared, for example, according to the method shown in Reaction Scheme I in which the HIV antigen-containing moiety is linked to the IRM moiety through R₁. In step (1) of Reaction Scheme I a compound of Formula III is reacted with a heterobifunctional cross-linker of Formula IV to provide a compound of II. R_{A} and R_{B} each contain a functional group that is selected to react with the other. For example, if R_{A} contains a primary amine, then a heterobifunctional cross-linker may be selected in which R_{B} contains an amine-reactive functional group such as an N-hydroxysulfosuccinimidyl ester. R_{A} and R_{B} may be selected so that they react to provide the desired linker group in the conjugate.

Methods for preparing compounds of Formula III where R_{A} contains a functional group are known. See for example, U.S. Pat. Nos. 4,689,338; 4,929,624; 5,268,376; 5,389,640; 5,352,784; 5,494,916; 4,988,815; 5,367,076; 5,175,296; 5,395,937; 5,741,908; 5,693,811; 6,069,149; 6,194,425; 6,331,539; 6,451,810; 6,525,064; 6,541,485; 6,545,016; 6,545,017; 6,573,273; 6,656,938; 6,660,747; 6,664,260; 6,667,312; 6,670,372; 6,677,349; and 6,683,088; U.S. patent Publication No. 2004/0010007; and International Patent Publication No. WO 04/058759.

Many heterobifunctional cross-linkers are known and many are commercially available. See for example, Hermanson, G. (1996), Bioconjugate Techniques, Academic Press, Chapter 5 “Heterobifunctional Cross-Linkers”, 229-285. The reaction generally can be carried out by combining a solution of the compound of Formula III in a suitable solvent such as N,N-dimethylformamide with a solution of the heterobifunctional cross-linker of Formula IV in a suitable solvent such as N,N-dimethylformamide. The reaction may be run at ambient temperature. The product of Formula II may then be isolated using conventional techniques.

In step (2) of Reaction Scheme I, a compound of Formula II that contains reactive group Z_{A} is reacted with the HIV antigen to provide the IRM-HIV conjugate of Formula I. The reaction generally can be carried out by combining a solution of the compound of Formula II in a suitable solvent such as dimethyl sulfoxide with a solution of the HIV antigen in a suitable buffer such as PBS. The reaction may be run at ambient temperature or at a reduced temperature (−4° C.). If Z_{A} is a photoreactive group such as a phenyl azide then the reaction mixture will be exposed to long wave UV light for a length of time adequate to effect cross-linking (e.g., 10-20 minutes). The average number of IRM moieties per HIV antigen moiety may be controlled by adjusting the amount of compound of Formula II used in the reaction. The IRM-HIV conjugate of Formula I may be isolated and purified using conventional techniques.

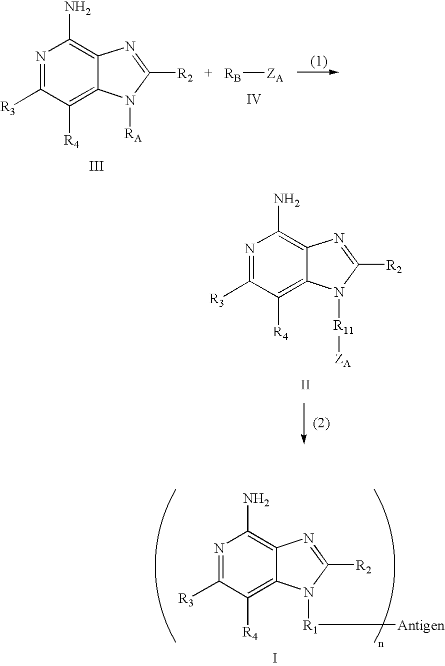

Alternatively, a compound of Formula II may be synthesized without using a heterobifunctional cross-linker. So long as the compound of Formula II contains the reactive group Z_{A}, it may be reacted with the HIV antigen using the method of step (2) above to provide an IRM-HIV conjugate.

As used herein, the terms “alkyl”, “alkenyl” and the prefix “alk-” include straight chain, branched chain, and cyclic groups, i.e. cycloalkyl and cycloalkenyl. Unless otherwise specified, these groups contain from 1 to 20 carbon atoms, with alkenyl groups containing from 2 to 20 carbon atoms. Preferred groups have a total of up to 10 carbon atoms. Cyclic groups can be monocyclic or polycyclic and preferably have from 3 to 10 ring carbon atoms. Exemplary cyclic groups include cyclopropyl, cyclopentyl, cyclohexyl, cyclopropylmethyl, and adamantyl.

The term “haloalkyl” is inclusive of groups that are substituted by one or more halogen atoms, including perfluorinated groups. This is also true of groups that include the prefix “halo-”. Examples of suitable haloalkyl groups are chloromethyl, trifluoromethyl, and the like.

The term “aryl” as used herein includes carbocyclic aromatic rings or ring systems. Examples of aryl groups include phenyl, naphthyl, biphenyl, fluorenyl and indenyl. The term “heteroaryl” includes aromatic rings or ring systems that contain at least one ring hetero atom (e.g., O, S, N). Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, and so on.

“Heterocyclyl” includes non-aromatic rings or ring systems that contain at least one ring hetero atom (e.g., O, S, N) and includes all of the fully saturated and partially unsaturated derivatives of the above mentioned heteroaryl groups. Exemplary heterocyclic groups include pyrrolidinyl, tetrahydrofuranyl, morpholinyl, thiomorpholinyl, piperidinyl, piperazinyl, thiazolidinyl, isothiazolidinyl, and imidazolidinyl.

The aryl, heteroaryl, and heterocyclyl groups can be unsubstituted or substituted by one or more substituents independently selected from the group consisting of alkyl, alkoxy, methylenedioxy, ethylenedioxy, alkylthio, haloalkyl, haloalkoxy, haloalkylthio, halogen, nitro, hydroxy, mercapto, cyano, carboxy, formyl, aryl, aryloxy, arylthio, arylalkoxy, arylalkylthio, heteroaryl, heteroaryloxy, heteroarylthio, heteroarylalkoxy, heteroarylalkylthio, amino, alkylamino, dialkylamino, heterocyclyl, heterocycloalkyl, alkylcarbonyl, alkenylcarbonyl, alkoxycarbonyl, haloalkylcarbonyl, haloalkoxycarbonyl, alkylthiocarbonyl, arylcarbonyl, heteroarylcarbonyl, aryloxycarbonyl, heteroaryloxycarbonyl, arylthiocarbonyl, heteroarylthiocarbonyl, alkanoyloxy, alkanoylthio, alkanoylamino, arylcarbonyloxy, arylcarbonythio, alkylaminosulfonyl, alkylsulfonyl, arylsulfonyl, heteroarylsulfonyl, aryldiazinyl, alkylsulfonylamino, arylsulfonylamino, arylalkylsulfonylamino, alkylcarbonylamino, alkenylcarbonylamino, arylcarbonylamino, arylalkylcarbonylamino, heteroarylcarbonylamino, heteroarylalkycarbonylamino, alkylsulfonylamino, alkenylsulfonylamino, arylsulfonylamino, arylalkylsulfonylamino, heteroarylsulfonylamino, heteroarylalkylsulfonylamino, alkylaminocarbonylamino, alkenylaminocarbonylamino, arylaminocarbonylamino, arylalkylaminocarbonylamino, heteroarylaminocarbonylamino, heteroarylalkylaminocarbonylamino and, in the case of heterocyclyl, oxo. If other groups are described as being “substituted” or “optionally substituted”, then those groups can also be substituted by one or more of the above-enumerated substituents.

Certain substituents are generally preferred. For example, preferred R₂ groups include hydrogen, alkyl groups having 1 to 4 carbon atoms (i.e., methyl, ethyl, propyl, isopropyl, n-butyl, sec-butyl, isobutyl, tert-butyl, and cyclopropylmethyl), and alkoxyalkyl groups (e.g., methoxyethyl and ethoxymethyl). Preferably R₃ and R₄ are independently hydrogen or methyl or R₃ and R₄ join together to form a benzene ring, a pyridine ring, a 6-membered saturated ring or a 6-membered saturated ring containing a nitrogen atom. One or more of these preferred substituents, if present, can be present in the compounds of the invention in any combination.

In some embodiments, an IRM-HIV conjugate may include a solid support structure to which both the HIV antigenic portion and the IRM portion are attached. In some embodiments, the IRM portion, HIV antigenic portion, or both may be covalently attached to the solid support using a linking group such as those described above. The solid support may include, for example, agarose beads, gold particles, and the like. The solid support may then be used to co-deliver the attached IRM portion and HIV antigenic portion to the appropriate target cell population. Methods for attaching IRMs to solid supports are described, for example, in U.S. patent Publication No. 2004/0258698 and U.S. patent Publication No. 2004/0202720. Methods for attaching biomolecules to solid supports are known in the art. Protocols for immobilizing biomolecules on solid supports are well known in the art and suitable reagents are available from commercial sources.

IRM-HIV compositions according to the present invention may contain chemical associations between the IRM portion and the HIV antigenic portion other than covalent coupling. For example, an IRM-HIV composition may include an affinity interaction between the HIV antigenic portion and the IRM portion. Avidin-biotin affinity represents one example of a non-covalent interaction that may be utilized to pair an HIV antigenic portion with an IRM portion. A biotin molecule may be chemically attached to an HIV antigen via one of a number of functional groups present on amino acids in, for example, a proteinaceous antigen (e.g., primary amines or sulfhydryl groups). An IRM portion may be conjugated to an avidin molecule by similar chemical means. The IRM portion and the HIV antigenic portion may then be paired by the avidin-biotin affinity interaction. Methods for biotinylating proteins and linking chemical groups to avidin are well known to one of skill in the art. Alternative affinity interactions that may be useful for making IRM-HIV compositions include, for example, antigen/antibody interactions, and glycoprotein/lectin interactions.

An IRM-HIV composition also may be formed by ionic interactions between an IRM portion and an HIV antigenic portion. For example, an IRM portion, an HIV antigenic portion, or both, may be chemically modified to contain oppositely charged components. The oppositely charged IRM portion and HIV antigenic portion may then be incubated together to allow for ionic interaction between the two entities. The resulting IRM-HIV composition may then be administered to a subject or a cell population, resulting in the co-delivery of both the IRM and the HIV antigen to the target cells.

As in the case of covalently linked IRM-HIV conjugates, IRM-HIV compositions in which the IRM portion and the HIV antigenic portion are paired non-covalently can include a solid support.

An IRM-HIV composition also may include a colloidal suspension. IRMs that are particularly useful for the preparation of a colloidal suspension are described in International Patent Publication No. WO 05/018555 and U.S. patent Publication No. 2004/0091491.

An IRM-HIV composition may be used to elicit an immune response from cells of the immune system in vitro or in vivo. Thus, an IRM-HIV composition may be useful as a component of a vaccine or as an immunostimulatory factor used in in vitro cell culture of T cells or B cells. Indeed, an IRM-HIV composition may be a more potent immunostimulatory factor than either the IRM portion or the HIV antigenic portion are capable of being if administered alone, or even if delivered together, but in an unpaired manner. When used to elicit an immune response in vitro, the immune cells activated in vitro may be reintroduced into a patient. Alternatively, factors secreted by the activated immune cells, e.g., antibodies, cytokines, and the like, may be collected for investigative, diagnostic, and/or therapeutic uses.

Unless otherwise noted, a host may be immunized in any suitable manner (e.g., subcutaneously, intraperitoneally, etc.). After a sufficient time to allow the host to generate an immune response to the IRM-HIV composition, immune cells appropriate for the immunization site are harvested. For example, lymph nodes may be harvested from a host that had been immunized subcutaneously. Spleen cells may be harvested from a host immunized peritoneally. For some hosts, cell harvesting may include sacrificing the hosts. In other cases, cell harvesting may include a biopsy or surgical removal of an appropriate tissue.

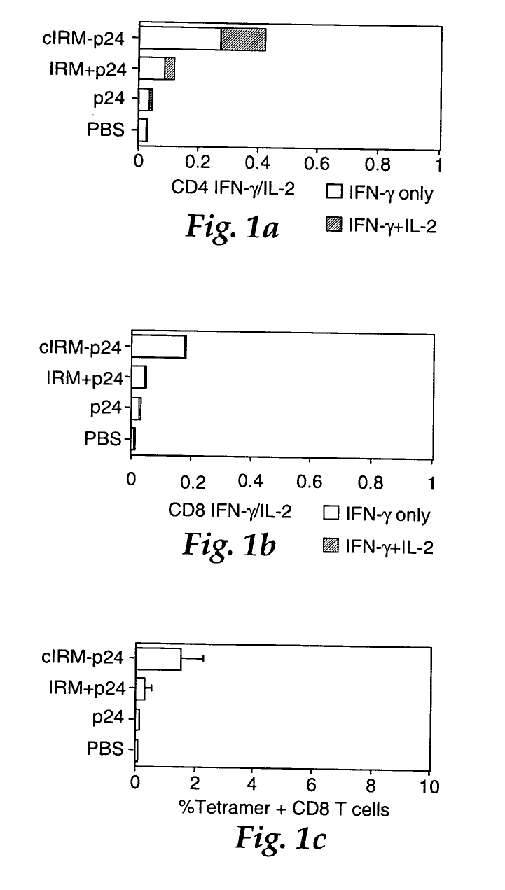

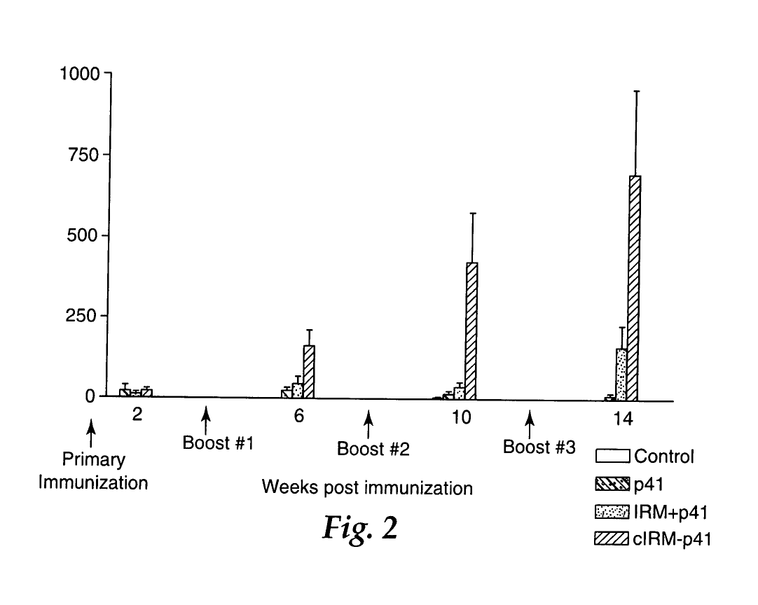

Immunizing a host with an IRM-HIV composition may be used to elicit an antigen-specific response in CD8⁺ cytotoxic T lymphocytes (CTLs). FIG. 1b and FIG. 1c show the generation of a CTL response by CD8⁺ T cells. The IRM-HIV composition induces a greater CTL response than does immunization with p24 alone or unpaired IRM and p24. FIG. 1c also shows that the IRM-HIV induces a larger population of antigen-specific CD8⁺ T cells. FIGS. 2, 3, 4a, and 4b demonstrate that similar results are obtained using a different HIV antigen, p41 Gag.

The CTL response generated by administering an IRM-HIV composition may provide therapeutic therapy to a subject infected with HIV. Alternatively, an IRM-HIV composition also may be administered prophylactically to provide a subject with a protective CTL immunity directed against a future HIV infection.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

The IRM portion of the IRM-HIV composition used in the following examples is N-[6-({2-[4-amino-2-(ethoxymethyl)-1H-imidazo[4,5-c]quinolin-1-yl]-1,1-dimethylethyl}amino)-6-oxohexyl]-4-azido-2-hydroxybenzamide, the synthesis of which is described in U.S. Published patent application No. 2004/0091491.

### Example 1

### Conjugation of IRM to HIV Gag

IRM was suspended in dimethyl sulfoxide (DMSO) to 10 mg/mL. HIV Gag p24 or HIV Gag p41 was suspended in phosphate buffered saline (PBS) to 1-2 mg/mL and the pH adjusted to >10.0 by the addition of NaOH. 500 μL of the HIV Gag solution (0.5-1.0 mg HIV Gag) was mixed with 50 μL of the IRM solution (500 μg IRM) in a single well of a 96 deep well (2 mL volume) polypropylene plate. The plate was placed on ice and a long wavelength UV light source was placed directly over the plate as close to the well containing the IRM/IV Gag mixture as possible. The mixture was irradiated for 2-5 minutes. The resulting conjugate was removed from the well and dialyzed against PBS to remove any unconjugated IRM. The conjugated IRM-HIV Gag was resuspended in PBS to a concentration of 500 μg/mL-1 mg/mL. The protein content of different batches of conjugate was determined by SDS-PAGE, and used to standardize the immunizations. Thus, doses of IRM-HIV Gag in the following examples are expressed in terms of the Gag protein provided in the dose.

### Example 2

Balb/c mice were immunized subcutaneously on Day 0 with either IRM-p24 Gag conjugate (cIRM-p24), unpaired IRM+p24 Gag (IRM+p24), p24 Gag (p24), or PBS. P24 Gag was administered in a dose of 10 μg, whether free or conjugated. Unpaired IRM, when administered, was administered in a dose of 17.5 μg.

The mice received booster immunizations at three weeks and six weeks after the initial immunization. At seven weeks after initial immunization, the percentage of CD4⁺ cells and CD8⁺ T cells expressing IFN-γ and IL-2 were determined by flow cytometry. FIG. 1a shows the T_{H}1 response, determined by detecting CD4⁺ cells expressing IFN-γ and IL-2. FIG. 1b shows the cytotoxic T lymphocyte (CTL) response, determined by detecting CD8⁺ T cells expressing IFN-γ and IL-2. FIG. 1c confirms the CTL response, determined by detecting CD8⁺ T cells stained with p24-specific tetramer.

### Example 3

Indian Rhesus macaques were immunized subcutaneously on Day 0 with p41 Gag protein (p41), unpaired IRM+p41 Gag protein (IRM+p41), IRM-p41 Gag protein conjugate (cIRM-p41), or PBS. P41 Gag protein was administered in a dose of 200 μg, whether free or conjugated. Unpaired IRM, when administered, was administered in a dose of 2 mg. Booster immunizations were administered at four weeks, eight weeks, and twelve weeks.

IFN-γ producing cells were measured by ELISPOT analysis at two weeks, six weeks, ten weeks, and fourteen weeks after initial immunization. Results are shown in FIG. 2.

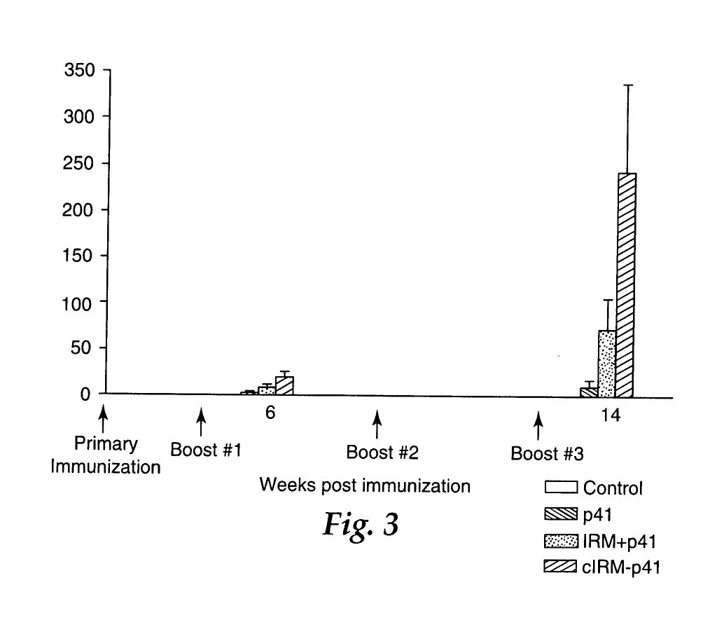

IL-2 producing cells were measured by ELISPOT analysis at six weeks and at fourteen weeks after initial immunization. Results are shown in FIG. 3.

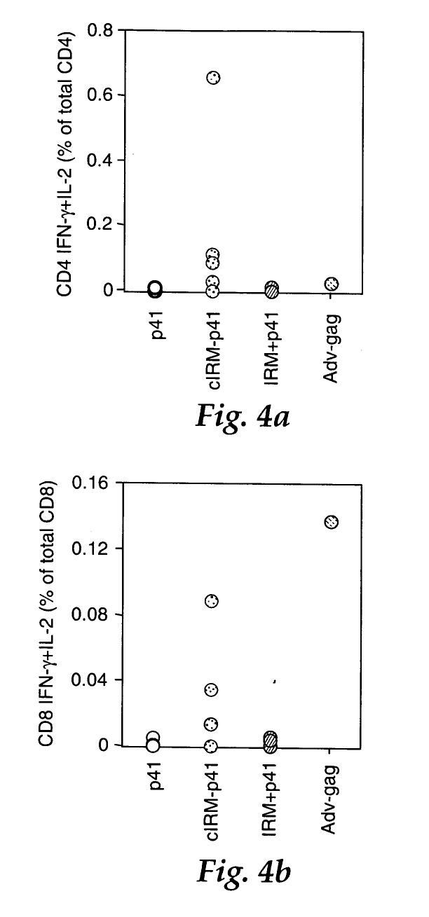

The percentage of CD4⁺ cells producing IFN-γ and IL-2 is shown in FIG. 4a. The percentage of CD8⁺ T cells producing IFN-γ and IL-2 is shown in FIG. 4b.

### Example 4

Indian Rhesus macaques were immunized as in Example 3 and serum was collected after the fourth immunization (i.e., at twelve weeks). 96-well plates were coated with HIV Gag protein at 4° C., washed three times with phosphate buffered saline (PBS)/Tween, and blocked with PBS/10% fetal calf serum (FCS). Serum samples were added to wells in serial dilutions and incubated at room temperature for two hours. After washing, horseradish peroxidase-conjugated anti-IgG (BD Biosciences Pharmingen, San Diego, Calif.) was added to each well and the plates incubated for one hour at room temperature. Plates were washed, then developed using TMB substrate-chromogen (DakoCytomation, Inc., Carpinteria, Calif.) according to manufacturer's instructions and read using a SpectraMax® Plus machine (Molecular Devices Corp., Sunnyvale, Calif.).

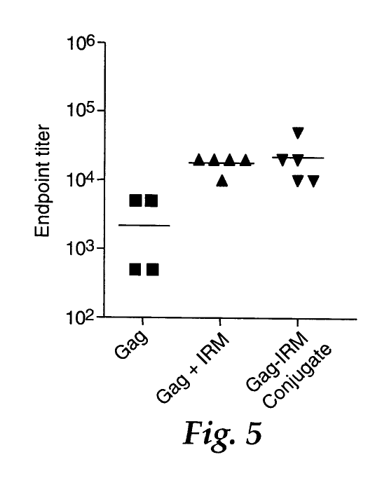

Results are shown in FIG. 5.

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

1. A composition of matter comprising:
an IRM portion; and
an HIV antigenic portion paired with the IRM portion.

2. The composition of claim 1 wherein the IRM portion is an agonist of at least human TLR7, or human TLR8.

3. The composition of claim 1 wherein the IRM portion comprises an imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

4. The composition of claim 1 wherein the IRM portion and the HIV antigenic portion are covalently conjugated.

5. The composition of claim 1 wherein the IRM portion and the HIV antigenic portion are paired by a physical or chemical association other than covalent conjugation that limits independent diffusion of the IRM portion with respect to the HIV antigenic portion.

6. The composition of claim 1 wherein the composition comprises a colloidal suspension.

7. The composition of claim 1 wherein the HIV antigenic portion comprises a Gag protein or polyprotein, an Env protein or polyprotein, a Pol protein or polyprotein, Nef, Pro, Rev, Tat, Vif, Vpr, Vpx, or an antigenic fragment thereof.

8. The composition of claim 7 wherein the HIV antigenic portion comprises two or more HIV antigens.
